(19) United States
(12) Patent Application Publication
(10) Pub. No.: US 2018/0311075 A1
(43) Pub. Date: Nov. 1, 2018

(54) SYSTEM AND A METHOD FOR REDUCING PRESSURE IN A PRESSURIZED CHAMBER

(71) Applicant: Camras Vision Inc., Research Triangle Park, NC (US)

(72) Inventors: Lucinda Camras, Durham, NC (US); Rolf Erik Ypma, Durham, NC (US)

(21) Appl. No.: 15/499,172

(22) Filed: Apr. 27, 2017

(51) Int. Cl. A61F 9/007

(52) U.S. Cl. CPC A61F 9/00781

(57) ABSTRACT

A system and associated method for reducing pressure are provided. The system includes a pressure-reducing device having an inlet tubular member defining an inlet port and adapted to engage a pressurized chamber having a fluid therein, and an outlet tubular member having a distal end defining an outlet port being adapted to extend to a surface external to the pressurized chamber. The pressure-reducing device channels the fluid to the surface external to the pressurized chamber via the outlet port. The system also includes manipulation tools configured to adjust one or more channels defined by the outlet tubular member to regulate a pressure within the pressurized chamber, the length of the one or more channels being proportional to the flow resistance imparted to or the backpressure on a fluid flowing from the inlet port to the outlet port by at least the outlet tubular member.

### BACKGROUND

### Field of the Disclosure

Aspects of the present disclosure are generally directed to a system and method for reducing pressure in a pressurized chamber. More particularly, the system and method are directed to reducing intraocular pressure.

### Description of Related Art

Glaucoma is a group of chronic optic nerve diseases and a leading cause of irreversible blindness. The major risk factor in glaucoma is elevated intraocular pressure due to improper drainage of aqueous humor from the eye. Reduction of intraocular pressure is the only proven treatment to stop the progression of vision loss by reducing stress on the optic nerve.

Standard glaucoma surgeries to reduce intraocular pressure, such as trabeculectomies and glaucoma drainage device implantation, tend to be lengthy and traumatic with unpredictable outcomes and complication rates of 20-60%. Implantable drainage devices function to drain excess aqueous humor from the eye, and installation of such a drainage device typically requires a surgical opening made in the sclera to reach the interior of the eye, in particular the anterior chamber or the posterior chamber. The drainage device is then inserted into the interior of the eye for conducting the aqueous humor to the subconjunctival space (with such a device herein referred to as a subconjunctival shunt), or externally of the conjunctiva (with such a device herein referred to as an external shunt).

A problem associated with subconjunctival shunts is potential scarring of the bleb in the subconjunctival space affecting its fibrous capsule formation around the outlet, which in many cases requires surgical revision that leads to additional risk of complications. Therefore, there is an ongoing search to identify and utilize alternate drainage sites to avoid many problems associated with bleb and fibrous capsule formations.

External shunts advantageously avoid bleb and fibrous capsule formation and the unpredictability of wound healing in the subconjunctival space. However, external shunts may not be capable of self-regulating or personalizing intraocular pressure. In certain cases, physicians may want to lower the intraocular pressure even further as one patient may cease vision loss with a pressure of 14 mmHg, while another patient may continue to lose vision with a pressure of 12 mmHg. Also, the pressure may increase over time due to clogging from proteins or other substances in the aqueous humor reducing permeability of the external shunt.

For the foregoing reasons there is a need for an improved system and method for reducing pressure in a pressurized chamber; specifically intraocular pressure.

### SUMMARY OF THE DISCLOSURE

The above and other needs are met by aspects of the present disclosure which, in one aspect, provides a system for reducing pressure. The system includes a pressure-reducing device including an inlet tubular member defining an inlet port, the inlet port being in communication with a central cavity defined by a housing and adapted to engage a pressurized chamber having a fluid therein, and an outlet tubular member having a distal end defining an outlet port, the outlet port being in communication with the central cavity via one or more channels defined by the outlet tubular member, the distal end of the outlet tubular member being adapted to extend to a surface external to the pressurized chamber, the pressure-reducing device receiving the fluid from the pressurized chamber via the inlet port and channeling the fluid to the surface external to the pressurized chamber via the central cavity and the outlet port. The system also includes a cutting tool configured to be capable of engaging the distal end of the outlet tubular member so as to adjust a length of the one or more channels defined by the outlet tubular member to regulate a pressure within the pressurized chamber, the length of the one or more channels being proportional to the flow resistance imparted to or the backpressure on a fluid flowing from the inlet port to the outlet port by at least the outlet tubular member.

In another aspect, a method for reducing pressure is provided. The method includes engaging an inlet port defined by an inlet tubular member of a pressure-reducing device with a pressurized chamber having a fluid therein, the pressure-reducing device comprising a central cavity defined by a housing, the central cavity being in communication with the inlet port, and including an outlet tubular member having a distal end defining an outlet port, the outlet port being in communication with the central cavity via one or more channels defined by the outlet tubular member, the distal end of the outlet tubular member extending to a surface external to the pressurized chamber, the pressure-reducing device being configured to receive the fluid from the pressurized chamber via the inlet port and to channel the fluid to the external surface via the central chamber and the outlet port. The method further includes adjusting a length of the one or more channels defined by the outlet tubular member to regulate a pressure within the pressurized chamber, the length of the one or more channels associated with the outlet tubular member being proportional to the flow resistance imparted to or the backpressure on the fluid flowing from the inlet port to the outlet port by at least the outlet tubular member.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended, namely to be combinable, unless the context of the disclosure clearly dictates otherwise.

### BRIEF DESCRIPTION OF THE DRAWINGS

For a more complete understanding of the present invention, reference should now be had to the embodiments shown in the accompanying drawings and described below. In the drawings:

FIG. 1A is a planar view of an embodiment of a pressure-reducing device including an outlet tubular member defining a single channel;

FIG. 1B is an exploded perspective view of the pressure-reducing device as shown in FIG. 1A;

FIG. 1C is a planar view of an embodiment of a pressure-reducing device including an outlet tubular member defining two channels;

FIG. 2 is a schematic of a measuring device for measuring flow resistance imparted to or backpressure on fluid flowing from an inlet port to an outlet port of a pressure-reducing device engaged with an outlet tubular member defining the outlet port;

FIG. 3A is a side view of a first embodiment of a cutting tool;

FIG. 3B is a detail perspective view of a cutting element, a cut limiter, and a guide element of the cutting tool as shown in FIG. 3A;

FIG. 3C is a front view of the cutting tool as shown in FIG. 3A in an open position;

FIG. 3D is a front view of the cutting tool as shown in FIG. 3A in a closed position;

FIG. 4A is a planar view of a second embodiment of a cutting tool;

FIGS. 4B-4D are detail perspective views of a cutting element and a cut limiter as shown in FIG. 4A in various positions relative to a guide element;

FIGS. 5A-5C are perspective views of various embodiments of an insertion tool;

FIGS. 6A-6B are various views of an embodiment of an adapter mechanism;

FIG. 6C is a side view of the adapter mechanism of FIGS. 6A-6B engaged with an outlet tubular member;

FIG. 6D is a cross-sectional view of the adapter mechanism and the outlet tubular member of FIG. 6C;

FIGS. 7A-7D are perspective views of various embodiments of an insert introduced in an outlet tubular member and defining one or more micro-channel;

FIG. 8 is a perspective view of a bore tool;

FIG. 9 is a perspective view of a compression tool engaged with an outlet tubular member;

FIGS. 10A-10E are cross-sections of various embodiments of an outlet tubular member defining two channels being opened and/or obstructed;

FIG. 11A is a perspective view of a first embodiment of a manipulation tool;

FIG. 11B is a detail planar view of the manipulation tool as shown in FIG. 11A;

FIG. 11C is a detail side view of the manipulation tool as shown in FIG. 11A;

FIG. 12A is a perspective view of a second embodiment of a manipulation tool having an adjustable loop arrangement in an extended configuration;

FIG. 12B is a perspective view of the manipulation tool as shown in FIG. 12A having the adjustable loop arrangement in a retracted configuration;

FIG. 13A is a perspective view of a third embodiment of a manipulation tool having an adjustable loop arrangement in an extended configuration;

FIG. 13B is a perspective view of the manipulation tool as shown in FIG. 13A having the adjustable loop arrangement in a retracted configuration;

FIG. 13C is a detail perspective view of the manipulation tool as shown in FIG. 13B;

FIG. 14A is a perspective view of a fourth embodiment of a manipulation tool;

FIG. 14B is a planar view of the manipulation tool as shown in FIG. 14A;

FIG. 14C is a detail planar view of the manipulation tool as shown in FIG. 14B;

FIG. 14D is a detail planar view of the manipulation tool as shown in FIG. 14C having a tubular member engaged therewith;

FIG. 15A is a side view of a fifth embodiment of a manipulation tool having a magnetized element;

FIG. 15B is a perspective view of a magnetic sleeve for cooperating with the magnetized element of the manipulation tool as shown in FIG. 15A; and

FIG. 16 is a method flow diagram of a method for reducing pressure.

### DETAILED DESCRIPTION

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

Certain terminology is used herein for convenience only and is not to be taken as a limitation on the scope of the disclosure. For example, words such as “upper,” “lower,” “left,” “right,” “horizontal,” “vertical,” “upward,” and “downward” merely describe the configuration shown in the figures. Indeed, the components may be oriented in any direction and the terminology, therefore, should be understood as encompassing such variations unless specified otherwise.

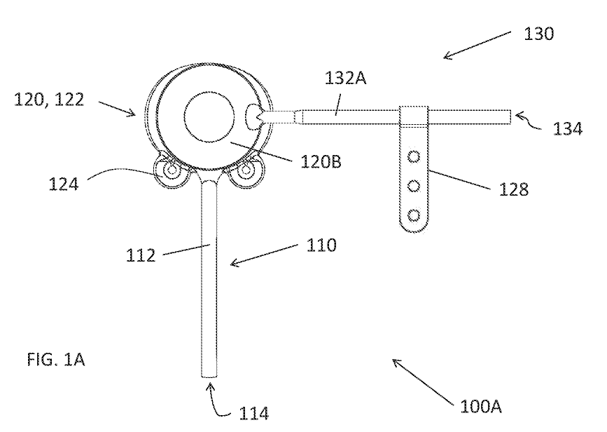

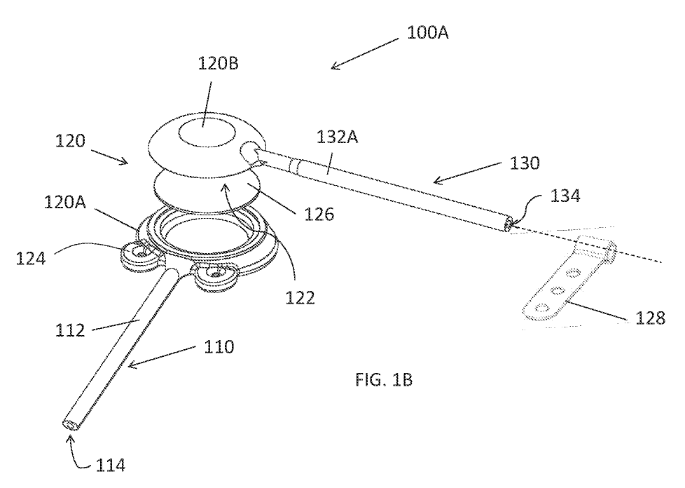

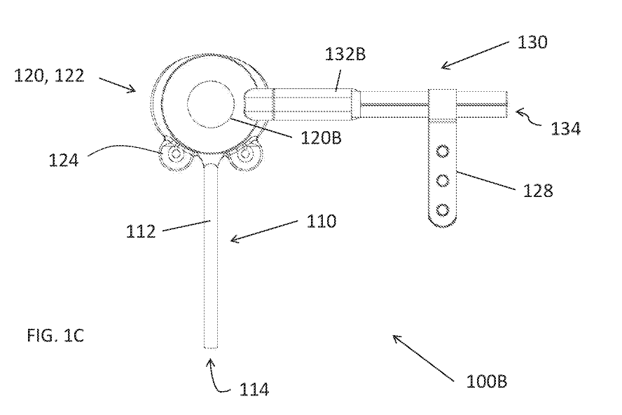

FIGS. 1A-1B schematically illustrate a first embodiment of a pressure-reducing device, generally designated as element 100A, according to one aspect of the present disclosure. The pressure-reducing device 100A, in some aspects, is implantable in an anterior chamber of an eye for reducing intraocular pressure.

The pressure-reducing device 100A generally comprises an inlet assembly 110 in communication with a central cavity 122 defined by a housing 120 and an outlet assembly 130 in communication with the central cavity 122. In some aspects, the inlet assembly 110 comprises an inlet tubular member 112 defining an inlet port 114. The inlet port 114 is adapted to engage a pressurized chamber (e.g., an anterior chamber of an eye) having fluid (e.g., aqueous humor) therein and thereby direct the fluid from the inlet port 114 to the central cavity 122. For example, in one instance, at least a portion of the inlet tubular member 112 of the pressure-reducing device 100A is implantable into the anterior chamber of an eye for draining aqueous humor therefrom. Representative configurations of such drainage devices of the general type disclosed herein are disclosed, for example, in U.S. Pat. No. 9,186,274 and U.S. Pat. No. 7,641,627, to Camras et al., each of which is incorporated herein by reference.

The inlet tubular member 112 of the pressure-reducing device 100A is substantially cylindrical and defines a hollow channel extending therethrough. As referenced throughout this description, a “channel” or “hollow channel” is one or more conduits defined by either the inlet tubular member 112 or an outlet tubular member 132A, 132B through a length thereof. The inlet tubular member 112 has a proximal end at which the inlet port 114 is defined and a distal end at which the inlet tubular member 112 is coupled to the central cavity 122. In some aspects, the proximal end of the inlet tubular member 112 is beveled or otherwise configured to facilitate entry of the proximal end of the inlet tubular member 112 into the pressurized chamber (e.g., the anterior chamber or other portion of the eye). In other aspects, the distal end of the inlet tubular member 112 defines a port that provides an outlet for fluid communication between the inlet tubular member 112 and the central cavity 122.

The hollow channel defined by the inlet tubular member 112 forms at least a portion of a flow path that permits the drainage of the fluid from the pressurized chamber to a surface external to the pressurized chamber. For example, the flow path permits drainage from the anterior chamber of the eye to a location or drainage site external to the anterior chamber. In this instance, the drainage site is an external ocular surface of the eye, such as the fornix or cul-de-sac region under the eyelid. In other instances, the drainage site includes another chamber within the eye, the subconjunctival space, the suprachoroidal space, or the like.

In some aspects, the inlet tubular member 112 has a length sufficient to provide the flow path between the pressurized chamber (e.g., the anterior chamber) and the external surface and to engage the housing 120 disposed on the external surface, thereby allowing the fluid to flow from the pressurized chamber through the hollow channel of the inlet tubular member 112 to the central cavity 122 defined by the housing 120. The fluid then flows from the central cavity 122 to an outlet tubular member 132A of the outlet assembly 130 and, in turn, to the external surface. For example, the aqueous humor is configured to flow from the anterior chamber of the eye through the inlet tubular member 112, through the central cavity 122 and the outlet assembly 130, and into the tear film associated with the eye when the pressure-reducing device 100A is implanted in or attached to the eye. For this purpose, the inlet tubular member 112 of the pressure-reducing device 100A has a minimum length, for example, of at least about 4 mm such that the housing 120 and outlet assembly 130 are positioned about the external surface (e.g., in the fornix or cul-de-sac region under the eyelid). In one aspect, the inlet tubular member 112 has a length of between about 4 mm and about 15 mm for adult humans. In other instances, the inlet tubular member 112 is provided in a standard length that is then cut to size by the surgeon prior to implantation. In use, in some aspects, the inlet tubular member 112 is implemented with the inlet port 114 being disposed in the pressurized chamber. For example, in use, the inlet tubular member 112 lies underneath the conjunctiva with the proximal end disposed in the anterior (or posterior) chamber of the eye. One skilled in the art will appreciate, however, that the dimensions and deployment location of the pressure-reducing device 100A varies considerably depending on the location to which the fluid drained from the pressurized chamber is directed.

In some aspects, an anchoring device or arrangement, such as one or more eyelet and/or bar is provided, adjacent the distal end of the inlet tubular member 112 and/or in engagement with the housing or head portion 120 of the pressure-reducing device 100A. For example, in one instance, the anchoring device comprises one or more eyelets 124 extending from a portion of an outer circumference of a first component of the housing 120A. In another example, one or more suture bars (not shown) extend from a portion of the outer surface of the inlet tubular member 112 or from an outer surface of the first component of the housing 120A. In these instances, the anchoring devices or arrangements are configured for contacting a surface external to the pressurized chamber (e.g., the sclera) when the pressure-reducing device 100A is implanted or engaged with, for example, the eye. More particularly, in this example, the one or more eyelets 124 are adapted for engaging the sclera and providing stability until and/or after biointegration of the inlet tubular member 112 and/or the housing/head portion 120 in the subconjunctival space.

In some aspects, a tab 128 is engageable with the outlet tubular member 132A (or outlet tube 132B, FIG. 1C) for adjustable positioning of the outlet tubular member 132A about the external surface of the eye. The tab 128 is configured to be removable with regard to the outlet tubular member 132A, or is permanent such that the tab 128 remains engaged with the outlet tubular member 132A throughout the lifetime of the device 100A. As illustrated in FIGS. 1A-1C, the tab 128 comprises a slideable engagement member that is configured to slide over or otherwise be engaged with an external surface of the outlet tubular member 132A away from the outlet port 134. In some aspects, the tab 128 is spaced apart from the outlet port 134 to aid in viewing and accessing the outlet tubular member 132A and outlet port 134 during exam. In this instance, the tab 128 is an alternative to suturing the outlet tubular member 132A to the eye as the suture may attract organic matter (e.g., mucus) to form about the outlet tubular member 132A and potentially occlude the outlet port 134. Accordingly, such an arrangement would lower the risk of blockage of the outlet port 134.

In some aspects, a planar surface extending from the slideable engagement member of the tab 128 is configured to conform to the external surface (e.g., the external surface of the eye). In such aspects, the planar surface defines one or more holes through which the tab 128 can be sutured to the eye to position the outlet tubular member 132A. For example, as illustrated in FIG. 1A, the planar surface defines three holes. However, in other examples, the planar surface is configured to be shortened to provide fewer holes based on positioning of the outlet tubular member 132A on the external surface.

The housing 120 defines the central cavity 122. The first component of the housing 120A is integral with, or otherwise attached to, the distal end of the inlet tubular member 112 such that the central cavity 122 is in fluid communication with the flow path defined by the inlet tubular member 112 so as to receive a flow of the fluid therefrom. A second component of the housing 120B is integral with, or otherwise attached to, a proximal end of the outlet tubular member 132A such that the central cavity 122 is in fluid communication with a flow path defined by the outlet tubular member 132A and is able to direct fluid therethrough.

In the illustrated aspects, the inlet assembly 110 and the outlet assembly 130 are formed separately from the other and cooperate, when assembled, to define the housing 120 which encompasses the central cavity 122 within an interior thereof. A filter element 126, in some aspects, is provided between the first component of the housing 120A and the second component of the housing 120B. The filter element 126 aids to prevent bacterial migration into the pressurized chamber (i.e., the anterior or posterior chamber of the eye), depending on a size of the pores of the filter element 126, and, in some aspects, the filter element 126 provides resistance to outflow from the pressurized chamber. In other aspects (not illustrated), the inlet assembly 110, the housing 120, and the outlet assembly 130 are integrally formed, separately or in combination.

According to some aspects, the first and/or second component of the housing 120A/120B is dome-shaped (or convex) to provide a substantially continuous transition surface from along an outer surface of the housing 120 to the surface external to the pressurized chamber (e.g., the convex surface of the eye, where the housing 120 is configured to lie on the conjunctiva). Such a configuration/shape of the first and/or second component of the housing 120A/120B results in the pressure-reducing device 100A being better tolerated upon implantation. More particularly, in this instance, the pressure-reducing device 100A is better tolerated if the device itself does not feel like a foreign object in the eye in relation to the eyelid.

In other instances, the housing 120 is placed or lies subconjunctivally in a patient. More particularly, for example, one or more components of the pressure-reducing device (e.g., outlet tube 132A, 132B) is exposed subconjunctivally, a length of which is variable depending on a placement of the housing 120. In such instances, the filter element 126 is optional and central cavity size may be reduced. One skilled in the art will also appreciate that other shapes of the first and/or second component of the housing 120A, 120B are also suitable and appropriate for providing a similar sensory perception for the user. For example, in some instances, a minimally protruding, substantially flat first and/or second component of the housing 120A/120B with rounded edges is able to be equally well tolerated. Other appropriate designs are determinable by those skilled in the art. For example, in other instances, the plan view of the housing 120 is round or ovular (see, e.g., FIG. 1A) or square or rectangular (not shown). The inner (convex) surface of the first and/or second component of the housing 120A/120B, in some examples, is flat or curved (or a combination of both), as appropriate, to correspond to the shape of the external surface.

As disclosed herein, the first and second components of the housing 120A, 120B are configured to form, when assembled, the housing 120 having the central cavity 122 defined therein. The central cavity 122, in communication with the inlet tubular member 112, is thus configured to receive the fluid from the pressurized chamber through the inlet port 114 of the inlet tubular member 112. The fluid received by the central cavity 122 is then able to be drained from the central cavity 122 to a drainage site disposed distally to the central cavity 122. As such, in some instances, the pressure-reducing device 100A further comprises the outlet assembly 130 including the outlet tubular member 132A having a proximal end in communication with the central cavity 122 and a distal end defining an outlet port 134. In some aspects, the proximal end of the outlet tubular member 132A is engaged with the housing 120 independently of the inlet tubular member 112, such that the distal end of the outlet tubular member 132A is spaced apart from the proximal end of the inlet tubular member 112. That is, the outlet tubular member 132A is configured to be engaged with the housing 120 separately from the inlet tubular member 112. The outlet tubular member 132A is, in some aspects, in communication with the central cavity 122 via one or more channels defined thereby. More particularly, the one or more channels defined by the outlet tubular member 132A, in some instances, longitudinally extend between the distal end defining the outlet port 134 and the proximal end in communication with the central cavity 122. For example, the outlet tubular member 132A defining one channel, two channels, three channels, etc., is configured such that each of the defined channels longitudinally extend for a selected length, and are disposed between the distal end defining the outlet port 134 and the proximal end in communication with the central cavity 122. In this way, a flow path is defined by cooperation between the one or more channels of the outlet tubular member 132A, the central cavity 122, and the inlet tubular member 112, such that fluid directed therethrough is channeled outwardly from the outlet port 134 onto the external surface.

In some aspects, a transverse/lateral cross-sectional shape of the outlet tubular member 132A, is other than circular, and is another suitable shape such as, for example, oval, square, trapezoidal, rectangular, or any combination thereof. In these aspects, the channels defined by the outlet tubular member 132A are similarly or differently shaped relative to the transverse/lateral cross-sectional shape of the outlet tubular member 132A. For example, where the transverse/lateral cross-sectional shape of the outlet tubular member 132A is circular, the channels defined by the outlet tubular member 132A are semi-circular as divided by a membrane laterally extending through the interior of the outlet tubular member 132A.

Regardless of shape, the cross-sectional size of the outlet tubular member 132A and/or the one or more channels defined thereby, in some instances, varies to selectively alter the fluid flow characteristics of the fluid. For example, in some cases, the one or more channels comprise a relatively small cross-sectional area in order to restrict the fluid flow of the fluid due to, for example, friction. In one aspect, the cross-sectional inner diameter of the outlet tubular member 132A ranges, for example, from about 200 to about 800 micrometers, while each of the one or more channels defined by the outlet tubular member 132A comprises a cross-sectional inner diameter of between about 25 and about 100 micrometers.

In other aspects, a minimum length, of the outlet tubular member 132A is, for example, at least about 4 millimeters. More particularly, the outlet tubular member 132A has a length of between about 6 and about 30 millimeters for adult humans. In other instances, the outlet tubular member 132A is provided in a standard length that is then cut to size by the surgeon prior to implantation.

The outlet port 134 is adapted to extend from the housing 120 to the surface external to the pressurized chamber. That is, the distal end of the outlet tubular member 132A defining the port 134 is spaced apart from the housing 120, such that the one or more channels defined by the outlet tubular member 132A are configured to receive the fluid from the central cavity 122 through the proximal end thereof and direct the fluid through the one or more channels and out of the outlet port 134 to an external surface disposed distally to, externally to, or otherwise away from the pressurized chamber and the housing 120. Alternatively, the outlet port 134 is adapted to extend away from the surface external to the pressurized chamber.

In some aspects, the outlet tubular member 132A defines a single channel in communication with the central cavity 122 for directing the fluid through the outlet port 134 to the external surface. In other aspects, however, the outlet tubular member 132A is configured such that the channel is bifurcated along at least a portion of the outlet tubular member 132A to form two (or more) channels (see, e.g., FIG. 1C).

FIG. 1C schematically illustrates a second embodiment of a pressure-reducing device, generally designated as element 100B, according to one aspect of the present disclosure. The pressure-reducing device 100B, in some aspects, is substantially similar in configuration to the first embodiment of the pressure-reducing device 100A illustrated in FIGS. 1A, 1B; however, the second embodiment of the pressure-reducing device 100B comprises an outlet tubular member 132B defining two channels extending at least partially therethrough.

More particularly, and as illustrated in FIG. 1C, the outlet tubular member 132B defines two channels separated by a bifurcation (e.g., a divider), the bifurcation extending from a distal end of the outlet tubular member 132B defining the outlet port 134 to a portion of the outlet tubular member 132B away from an opposing, proximal end of the outlet tubular member 132B. In this manner, the flow path defined by the outlet tubular member 132B changes from a single channel to two channels at the bifurcated portion of the outlet tubular member 132B. In some aspects, an inner diameter of each of the two channels is less than an inner diameter of the single channel. In some further aspects, an inner diameter of each of the two channels varies along a length of each channel. In other aspects, and not illustrated herein, the bifurcation extends from the distal end of the outlet tubular member 132B to the proximal end of the outlet tubular member 132B, such that the outlet tubular member defines two channels extending through the length of the outlet tubular member 132B.

Accordingly, the pressure-reducing device 100A, 100B is configured for implantation in, for example, an eye, in order to direct fluid from the pressurized chamber (e.g., the anterior chamber) to a surface external thereto and thereby reduce pressure within the pressurized chamber. However, in some instances the implanted pressure-reducing device 100A, 100B requires adjustment or manipulation to further reduce, increase, or otherwise regulate the pressure within the pressurized chamber. For example, in some aspects, the pressure-reducing device 100A, 100B requires adjustment of a length of the one or more channels defined by the outlet tubular member 132A, 132B so as to regulate a pressure within the pressurized chamber. That is, the length of the one or more channels of the outlet tubular member 132A, 132B is proportional to the flow resistance imparted to or the backpressure on the fluid flowing from the inlet port 114 to the outlet port 134 by at least the outlet tubular member 132A, 132B. In other examples, the pressure-reducing device 100A, 100B requires adjustment of a cross-sectional area of the one or more channels defined by the outlet tubular member 132A, 132B, the cross-sectional area of the one or more channels of the outlet tubular member 132A, 132B also being proportional to the flow resistance imparted to or the backpressure on the fluid flowing from the inlet port 114 to the outlet port 134 by at least the outlet tubular member 132A, 132B. Other methods of manipulating the implanted pressure-reducing device 100A, 100B to further reduce or increase pressure include opening additional channels within the outlet tubular member 132A, 132B, removing lateral portions of the outlet tubular member 132A, 132B, occluding or obstructing one or more channels within the outlet tubular member 132A, 132B, etc.

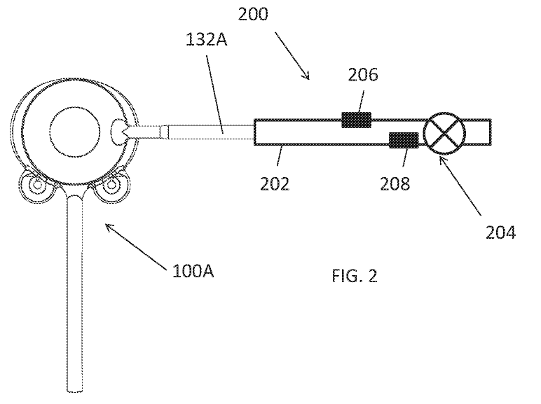

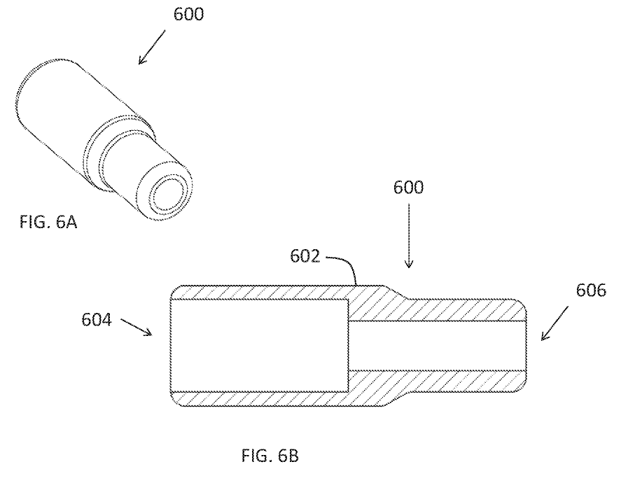

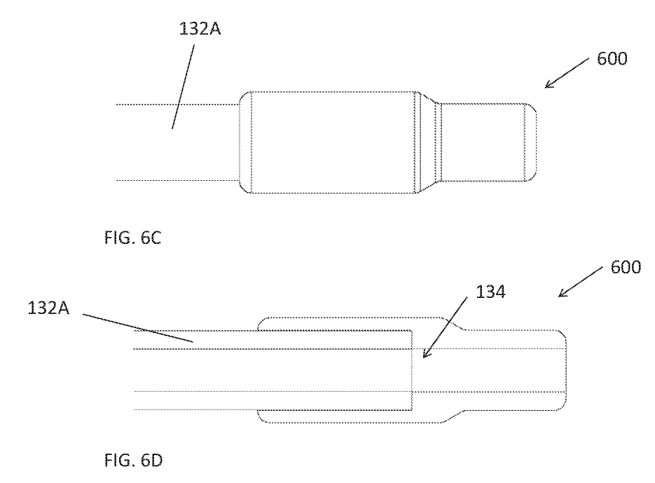

In some aspects, and as illustrated in FIG. 2, in order to determine whether the implanted pressure-reducing device 100A, 100B requires any adjustment or manipulation, a measuring device, generally designated 200, is utilized. The measuring device 200 is, in some aspects, a flow gauge, a pressure gauge, and/or a combination thereof that is configured to be capable of engaging the pressure-reducing device 100A, 100B about the housing 120 or the outlet tubular member 132A, 132B for measuring the flow resistance imparted to fluid or the backpressure on the fluid flowing from the inlet port 114 to the outlet port 134. For example, and as illustrated in FIG. 2, the measuring device 200 comprises a longitudinally extending tubular member 202 configured with an inner diameter greater than an outer diameter of the outlet tubular member 132A of the pressure-reducing device 100A. In this example, the longitudinally extending tubular member 202 of the measuring device 200 is configured to engage with the outlet tubular member 132A by cannulation or by threading the outlet 134 defined by the outlet tubular member 132A into a port defined by the longitudinally extending tubular member 202. In some aspects, an adapter mechanism (e.g., 600, FIGS. 6A-6D) is used for engagement between the outlet tubular member 132A and the measuring device 200.

In some aspects, the longitudinally extending tubular member 202 includes a valve 204 configured to open and close and, thereby, occlude or allow fluid flow along a flow path defined by the longitudinally extending tubular member 202 upon engagement with the outlet tubular member 132A. As illustrated in FIG. 2, one or more sensors are also provided with the measuring device 200 to obtain the flow measurements or the pressure measurements of the fluid. For example, a flow sensor 206 for measuring flow rate and/or a pressure sensor 208 for measuring backpressure are provided in-line with the outlet tubular member 132A. In such examples, the pressure sensor 208 is engaged with the valve 202 to determine a pressure of the pressurized chamber (e.g., intraocular pressure) when the valve 202 is closed. The flow measurements and/or the pressure measurements ascertained by the measurement device 200, in some aspects, are configured to be collected or transmitted to a computing platform (e.g., a data acquisition box) either wirelessly or in wired connection with the measuring device 200.

In other aspects, the intraocular pressure or backpressure is measured by using tonometry or by creating a closed system and cannulating the outlet tubular member 132A with a pressure sensor. The pressure sensor may be contained in a fluid collection vial. When trying to assess the flow rate through the device an open system is created to allow fluid to fill over a certain period of time to assess volume (e.g., microliter) per time (e.g., a minute), and the like. Other methods and systems for measuring characteristics of the fluid relative to the pressure-reducing device 100A, 100B known to those of skill in the art are also contemplated.

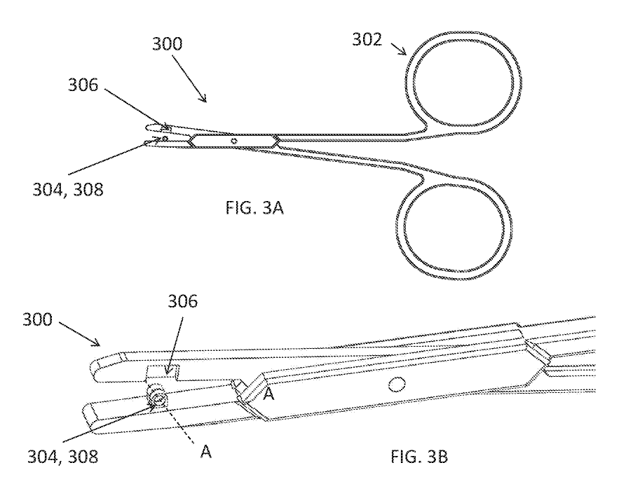

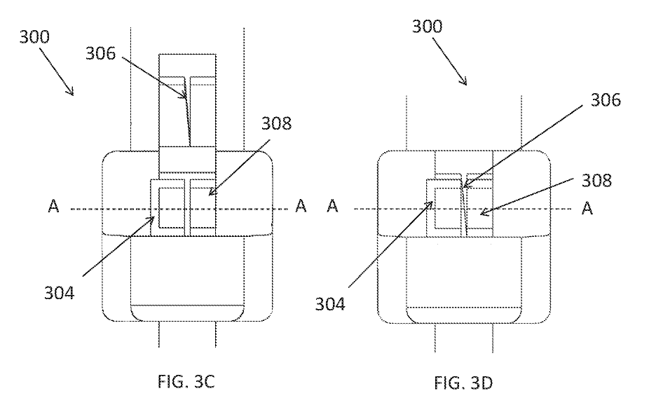

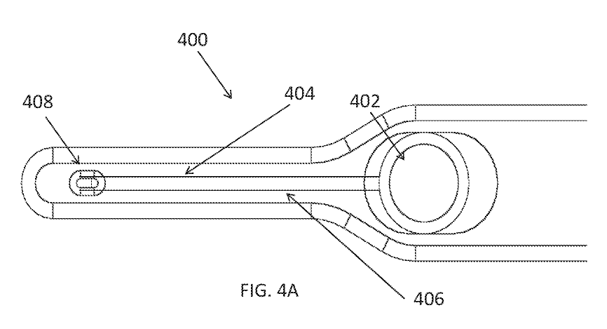

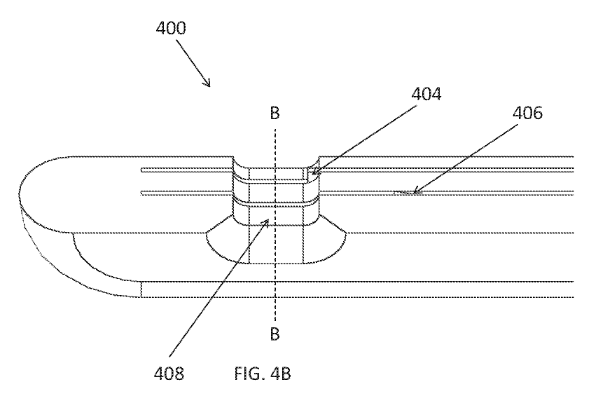

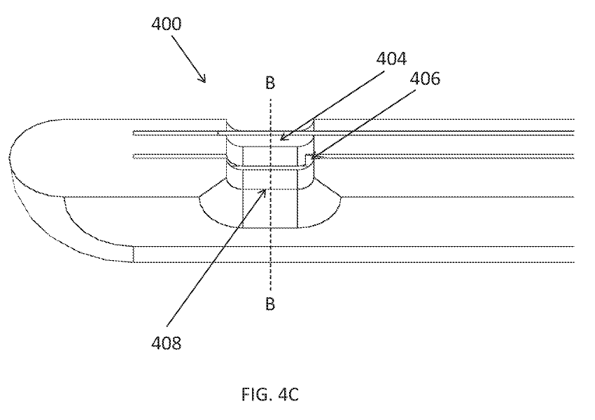

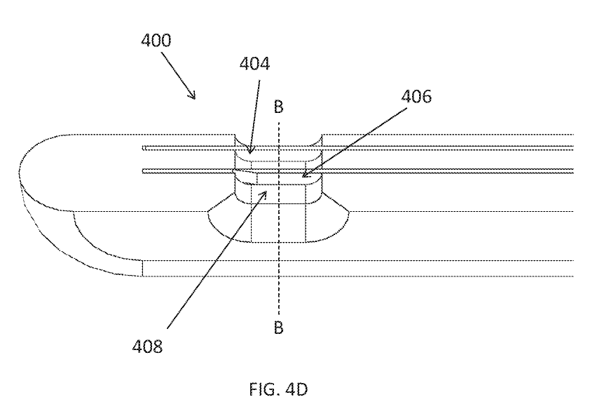

Upon determining the backpressure on and/or the flow relative to the pressure-reducing device 100A, 100B, a determination is made as to whether the pressure-reducing device 100A, 100B should be adjusted or manipulated in order to reduce or increase the backpressure and/or flow resistance. Accordingly, and now referring to FIGS. 3A-3D and 4A-4D, two exemplary embodiments of cutting tools capable of adjusting a length of the one or more channels defined by the outlet tubular member 132A, 132B are provided. While only two such cutting tools are described herein, this disclosure is in no way limited to cutting tools as described by these specific embodiments.

FIGS. 3A-3D illustrate a first exemplary embodiment of a cutting tool, generally designated 300, configured to be capable of engaging the distal end of an outlet tubular member 132A, 132B (see, e.g., outlet tubular member 132A, 132B, FIGS. 1A-1C) so as to adjust a length of the one or more channels defined by the outlet tubular member 132A, 132B to regulate a pressure within the pressurized chamber. As illustrated in FIG. 3A, the cutting tool 300 is similar to a pair of scissors that cuts in response to pressure applied to the handles 302 to bring them into close proximity.

In some aspects, the cutting tool 300 comprises a cut limiter 304 capable of engaging the distal end of the outlet tubular member 132A, 132B and a cutting element 306 spaced apart from the cut limiter 304 and capable of engaging the outlet tubular member 132A, 132B longitudinally therealong away from the distal end. Such a configuration, for example, limits an amount of the length of the outlet tubular member 132A, 132B (and thus the one or more channels defined thereby) that can be cut by the cutting tool 300 in one cut. For example, the cut limiter 304 and the cutting element 306 are arranged such that no more than about 1 millimeter in length is cut from the distal end of the outlet tubular member 132A, 132B.

In some aspects, the cutting tool 300 further comprises a guide element 308. For example, and as illustrated in FIGS. 3A-3D, the guide element 308 is aligned with and spaced apart from the cut limiter 304 along an axis A-A. In some aspects, and as illustrated in FIG. 3C, in an open position of the cutting tool 300, the guide element 308 is configured to receive the distal end of the outlet tubular member 132A, 132B therethrough with the distal end of the outlet tubular member 132A, 132B extending up to the cut limiter 304. In other aspects, and as illustrated in FIG. 3D, in a closed position of the cutting tool 300, the cutting element 306 is configured to extend between the guide element 308 and the cut limiter 304 to cut a limited portion of the outlet tubular member 132A, 132B along the length thereof. In other aspects, the cut limiter 304 is removable so as to remove any limitation on an amount of the length of the outlet tubular member 132A, 132B removable by the cutting tool 300 in one cut.

FIGS. 4A-4D illustrate a second exemplary embodiment of a cutting tool, generally designated 400. As illustrated in FIG. 4A, the cutting tool 400 is similar to a conventional cigar cutter that cuts by pressure applied to an actuator 402. In some aspects, the cutting tool 400 comprises a cut limiter 404 capable of engaging the distal end of the outlet tubular member 132A, 132B and a cutting element 406 spaced apart from the cut limiter 404 and capable of engaging the outlet tubular member 132A, 132B longitudinally therealong away from the distal end. Such a configuration, for example, limits an amount of the length of the outlet tubular member 132A, 132B (and thus the one or more channels defined thereby) that can be cut by the cutting tool 400 in one cut. For example, the cut limiter 404 and the cutting element 406 are arranged such that no more than about 1 millimeter in length is cut from the distal end of the outlet tubular member 132A, 132B.

In some aspects, the cutting tool 400 further comprises a guide element 408. For example, and as illustrated in FIGS. 4B-4D, the guide element 408 is contoured so as to guide the outlet tubular member 132A, 132B along an axis B-B into engagement with one or both of the cut limiter 404 and the cutting element 406. In some aspects, one of the cut limiter 404 and the cutting element 406 are laterally movable relative to the axis B-B in response to actuation by the actuator 402. In these aspects, the cut limiter 404 and/or the cutting element 406 are coupled to the actuator such that actuation of the actuator results in lateral movement of one or both of the cut limiter 404 and the cutting element 406 relative to the axis B-B.

FIG. 4B illustrates that the actuator (e.g., 402, FIG. 4A), upon partial actuation thereof, is configured to laterally move the cut limiter 404 into partial axial alignment with the guide element 408 prior to laterally moving the cutting element 406 to extend relative to the cut limiter 404 to cut the outlet tubular member along the length thereof. Notably, the cut limiter 404 is configured, in this aspect, to be independently actuated from the cutting element 406 so that actuation of the cut limiter 404 does not actuate the cutting element 406. That is, in FIG. 4B, the cutting element 406 is in a retracted position, while the cut limiter 404 is in a partially extended position.

FIG. 4C illustrates the cut limiter 404 in a fully extended position so that the cut limiter 404 is configured to limit an amount of the length of the outlet tubular member 132A, 132B cut from the distal end thereof by the cutting tool 400 in one cut. In this manner, the actuator, upon full actuation thereof relative to the cut limiter 404, is configured to laterally move the cut limiter 404 into full axial alignment with the guide element 408. Likewise in FIG. 4C, the actuator, upon partial actuation thereof, is configured to laterally move the cutting element 406 into partial axial alignment with the guide element 408, such that the cutting element 406 is partially extended relative to the cut limiter 404.

FIG. 4D illustrates both the cut limiter 404 and the cutting element 406 in the fully extended position. More particularly, the actuator, upon full actuation thereof relative to the cutting element 406, is configured to laterally move the cutting element 406 into full axial alignment with the guide element 408 and the cut limiter 404 to cut a limited portion of the length of the outlet tubular member 132A, 132B from the distal end thereof. In other aspects, the cut limiter 404 is removable so as to remove any limitation on an amount of the length of the outlet tubular member 132A, 132B removable by the cutting tool 400 in one cut.

Alternatively, for example, the actuator 402, upon actuation thereof, is configured to laterally move the cut limiter 404 into axial alignment with the guide element 408 substantially simultaneously with laterally moving the cutting element 406 to fully extend.

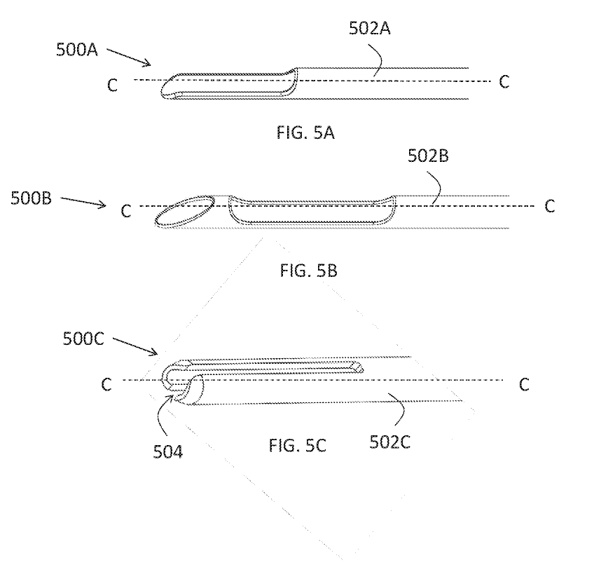

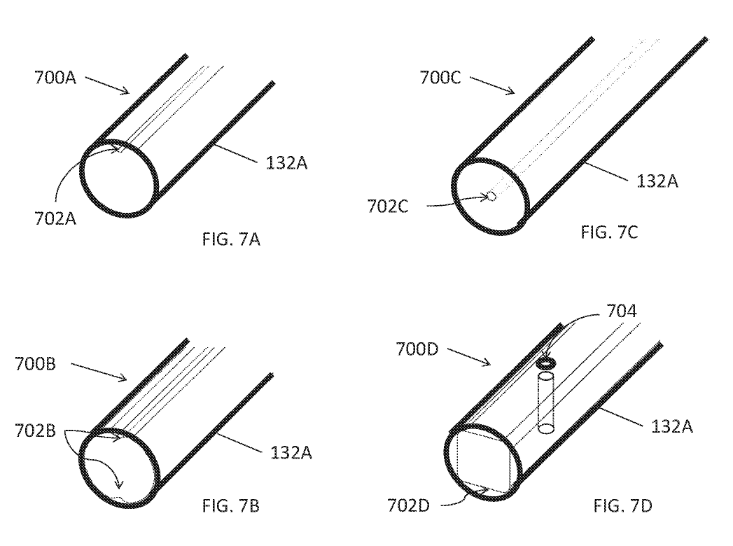

Now referring to FIGS. 5A-5C three exemplary embodiments of insertion tools 500A-500C each capable of inserting an insert into the one or more channels defined by the outlet tubular member 132A, 132B to increase or reduce a cross-section thereof are provided. While only three such insertion tools are described herein, this disclosure is in no way limited to insertion tools as described by these specific embodiments. Notably, the inserts described with regard to the insertion tools illustrated in FIGS. 5A-5C are described more fully with regard to FIGS. 7A-7D.

Generally, the insertion tools 500A-500C are configured to be capable of engaging the distal end of the outlet tubular member 132A, 132B so as to introduce an insert (see, e.g., FIGS. 7A-7D) into the outlet port 134. Each of the insertion tools 500A-500C comprises a cannula 502A-502C having a distal end insertable into the one or more channels defined by the outlet tubular member 132A, 132B axially through the outlet port 134. In these instances, the cannulas 502A-502C of the insertion tools are each configured to receive an insert therein.

FIGS. 5A-5C each illustrate different configurations of the cannulas 502A-502C. For example, as FIG. 5A illustrates, in one aspect the distal end of the cannula 502A is oblique or otherwise angled relative to a longitudinal axis C-C of the cannula 502A. FIG. 5B illustrates the distal end of the cannula 502B configured to taper away from the longitudinal axis C-C of the cannula 502B, such that a cut-out or opening is defined within an outer surface of the cannula 502B. FIG. 5C illustrates the distal end of the cannula 502C defining diametrically opposed longitudinal slots 504 relative to the longitudinal axis C-C of the cannula 502C extending from the distal end thereof. As illustrated in FIG. 5C, there are two diametrically opposed longitudinal slots 504 although there may be multiples thereof, such as four slots, eight slots, etc.

In some aspects, the cannula 502A-502C is made of a compressible and flexible material, such that pressure from a user's hands along the longitudinal axis C-C of the cannula 502A-502C acts to introduce the insert into the one or more channels defined by the outlet tubular member. Otherwise, where the cannula 502A-502C comprises a trocar, an actuation mechanism engaged with the trocar acts to introduce the insert into the one or more channels defined by the outlet tubular member. Likewise, in some aspects, the cannula 502A-502C is configured to be withdrawn from the one or more channels defined by the outlet tubular member 132A, 132B substantially simultaneously with axially moving the insert along the cannula 502A-502C and through the distal end thereof to introduce the insert into the one or more channels defined by the outlet tubular member 132A, 132B. For example, the cannula 502A-502C is configured to be withdrawn from the one or more channels defined by the outlet tubular member 132A, 132B via suction applied to a proximal end of the cannula 502A-502C.

In other aspects, and referring to FIGS. 6A-6D, an adapter mechanism 600 for extending the outlet tubular member, where the outlet tubular member has been reduced in length, is provided. More particularly, where the outlet tubular member is cut by a cutting tool, the adapter mechanism 600 is configured to temporarily be engaged with an outlet port of the outlet tubular member to provide increased space for easier access to the shortened outlet tubular member for various purposes (e.g., cannulation, insert insertion, etc.)

The adapter mechanism 600 comprises, in some aspects, a longitudinally extending tubular body 602 having a proximal end defining a first port 604 configured to be engaged with a distal end of the outlet tubular member and an opposing distal end defining a second port 606. Thus, a flow path is defined between the first port 604 and the second port 606 of the adapter mechanism such that fluid, inserts, tools, etc., are introduceable through the second port 606 to the outlet port of the outlet tubular member by way of the first port 604. For example, an insertion tool (e.g., cannula 500A-500C, FIGS. 5A-5C) is configured to introduce the insert received therein through the second port 606 of the adapter mechanism 600 and into the one or more channels defined by the outlet tubular member through the first port 604 of the adapter mechanism 600.

In some aspects, and as illustrated in FIG. 6D, the distal end defining the second port 606 comprises an inner diameter smaller than an inner diameter of the proximal end defining the first port 604 and substantially a same size as an inner diameter of the outlet port 134 defined by the outlet tubular member 132A. In other aspects, not shown, the distal end defining the second port 606 comprises an inner diameter smaller than an inner diameter of the proximal end defining the first port 604 and smaller than the inner diameter of the outlet port 134 defined by the outlet tubular member 132A. In this instance, the adapter mechanism 600 is configured to provide resistance to the fluid flowing from the outlet port 134 defined by the outlet tubular member 132A by reducing the flow cross-section.

In this manner, and referring to FIGS. 7A-7D, various exemplary embodiments of an insert insertable in one or more channels of a tubular member 132A are illustrated. In some aspects, for example, the insert is receivable in a single channel defined by the outlet tubular member 132A. In this instance, an insertion tool (e.g., tool 500A-500C, FIGS. 5A-5C) is capable of receiving the insert and subsequently inserting the insert into the single channel defined by the outlet tubular member 132A. Otherwise, where there is more than one channel defined by the outlet tubular member (e.g., outlet tubular member 132B, FIG. 1C), an insert is capable of being inserted into each channel, as needed.

In some aspects, the insert is configured to define one or more micro-channels extending axially between opposed longitudinal ends of the insert. As used herein, a “micro-channel” refers specifically to one or more longitudinal grooves, bores, chamfers, etc., extending axially between opposed longitudinal ends of the insert. As one of ordinary skill in the art understands, the more micro-channels that are formed within the insert, the more that flow resistance will decrease, such that backpressure is reduced. For example, in FIG. 7A, an insert 700A comprises a radially outermost surface that is longitudinally fluted and defines a micro-channel 702A formed as a single groove extending between opposed longitudinal ends of the insert 700A. In this manner, the micro-channel 702A is configured to cooperate with the channel defined by the outlet tubular member 132A to define a flow channel therebetween.

In another example, in FIG. 7B, two micro-channels are formed in an insert 700B. The insert 700B, in some aspects, comprises a radially outermost surface that is longitudinally fluted and defines two micro-channels 702B formed as two diametrically opposed grooves extending between opposed longitudinal ends of the insert 700B. In this manner, each of the diametrically opposed micro-channels 702B is configured to cooperate with the channel defined by the outlet tubular member 132A to define a flow channel therebetween. In other aspects, more than one pair of diametrically opposed micro-channels 702B (e.g., two pairs, three pairs, etc.,) is configured to be formed in the insert 700B.

In another example, in FIG. 7C, an insert 700C comprising a micro-channel 702C formed as a bore defined along a central axis of the insert 700C, is illustrated. The micro-channel 702C extends, in some aspects, through a length of the insert 700C between opposed longitudinal ends thereof. In other aspects, more than one micro-channel 702C is configured to be formed in the insert 700C.

In a still further example, in FIG. 7D, an insert 700D comprising a radially outermost surface that defines micro-channels formed from chamfers 702D is illustrated. In this instance, for example, the micro-channels 702D are formed at regularly spaced intervals (e.g., 90 degree intervals) around the radially outermost surface of the insert 700D and extend between opposed longitudinal ends of the insert 700D. In this manner, the micro-channels 702D spaced apart as illustrated in FIG. 7D result in an insert 700D having a square cross-section with rounded corners. Thus, each of micro-channels 702D is configured to cooperate with the channel defined by the outlet tubular member 132A to define a flow channel therebetween. In other aspects, the micro-channels 702D are spaced at 45 degree intervals, 180 degree intervals, etc., to form an insert 700D having an octagonal cross-section, a triangular cross-section, etc.

The insert 700D comprises, in some instances, a bore or hole 704 defined laterally through the circumferential surface of the outlet tubular member 132A and through a circumferential surface of the insert 700D, is an alternative to adjusting a length of the outlet tubular member 132A, for example, by cutting. In other aspects, more than one bore 704 is configured to be formed in the insert 700D.

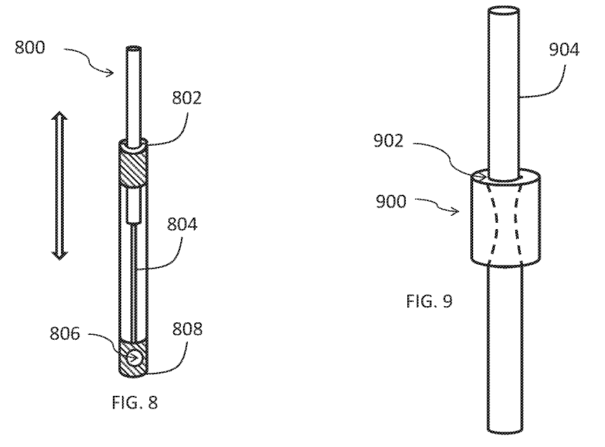

FIG. 8 illustrates an exemplary bore tool for forming a hole or bore laterally through an outlet tubular member (e.g., 132A, 132B, FIGS. 1A-1C) and/or an insert (e.g., 700A-700D, FIGS. 7A-7D) inserted therein. As illustrated in FIG. 8, for example, the boring tool comprises an awl 800 having a body portion 802, a boring element 804, a guide element 806, and a limiting region 808. In this example, the body portion 802 is configured to receive the boring element 804 within an interior cavity defined thereby, the boring element 804 being actuatable along a longitudinal axis defined by the body portion 802. The guide element 806 is defined by the body portion 802 as a laterally extending port relative to the longitudinal axis of the body portion 802. The guide element 806 is configured with a diameter larger than an exterior diameter of the outlet tubular member and the insert, so that the outlet tubular member is receiveable therethrough. Actuation of the bore tool 800 results in the boring element 804 extending laterally through the surface of the outlet tubular member and/or insert so as to form an outlet port laterally through the circumferential surface of the outlet tubular member.

In some aspects, a limiting region 808 is defined about the body portion 802 towards an end of the body portion 802 at which the guide element 806 is provided. The limiting region 808 is configured as a mechanism to prevent the boring element 804 from over-extending along the longitudinal axis of the body portion 802. Accordingly, the bore tool 800 is configured to form an outlet port supplemental to the outlet port 134 as illustrated in FIGS. 1A-1C. In this manner, the supplemental outlet port reduces a length of the one or more channels defined by the outlet tubular member as an alternative to cutting or otherwise adjusting a length of the outlet tubular member, itself.

In some aspects, where an insert 700A-D is inserted within the outlet tubular member 132A, such as those provided in FIGS. 7A-7D, the bore tool 800 is configured to form the supplemental outlet port laterally through the wall of the outlet tubular member as well as a circumferential surface of the insert. For example and referring back to FIG. 7D, the hole 704 is formed by the boring element 804 of the bore tool 800 laterally through the circumferential surface of the insert 700D and the outlet tubular member 132A as an alternative to cutting or otherwise adjusting a length of the outlet tubular member, itself.

In some aspects, a cross-sectional area of the one or more channels of the outlet tubular member 132A, 132B is configured to be reduced by a compression tool. FIG. 9 illustrates an exemplary embodiment of a compression tool, which as illustrated, is a compression sheath 900. The compression sheath 900 defines, in some aspects, a lumen 902. In this instance, the lumen 902 of the compression sheath 900 is configured to be capable of engaging an outlet tubular member, such as outlet tubular member 904 configured similarly to outlet tubular member 132A in FIGS. 1A, 1B, and extending about and compressing a circumferential surface of the outlet tubular member 904 to reduce the cross-sectional area of the channel defined thereby. The lumen 902 of the compression sheath 900 is capable, in some aspects, of being disengaged from the outlet tubular member 904 in order to return the cross-sectional area of the channel defined by the outlet tubular member 904 to a previous non-compressed state. In some aspects, the compression sheath 900 is configured to engage an outlet tubular member defining more than one channel (e.g., outlet tubular member 132B, FIG. 1C) in order to reduce the cross-section area of each of the more than one channel defined by the outlet tubular member. Other embodiments of a compression tool include, for example, a clamp, a ring, etc.

In other aspects, compression is achievable by tying a suture (not shown) about the sheath 900 or about the outlet tubular member 132, directly. For example, a suture is usable to extend around the circumferential surface of the outlet tubular member at least one time and subsequently be tightened so as to reduce the cross-sectional area of the one or more channels. Accordingly, the reduced cross-sectional area of the outlet tubular member increases the flow resistance imparted to the fluid or the backpressure on the fluid. The suture is capable, in some aspects, of being removed (e.g., cut) from the outlet tubular member in order to return the cross-sectional area of the one or more channels defined by the outlet tubular member to a previous, non-compressed state.

In some aspects, flow resistance and/or pressure is able to be adjusted by opening or occluding one or more of the channels of the outlet tubular member. For example, for an outlet tubular member defining multiple channels will have a proportional relationship to the number of channels opened and the pressure reduction based on the decrease in flow resistance. Likewise, for example, for an outlet tubular member defining two or more channels (e.g., outlet tubular member 132B, FIG. 1C), opening or occluding one or more of the channels will have a similar effect.

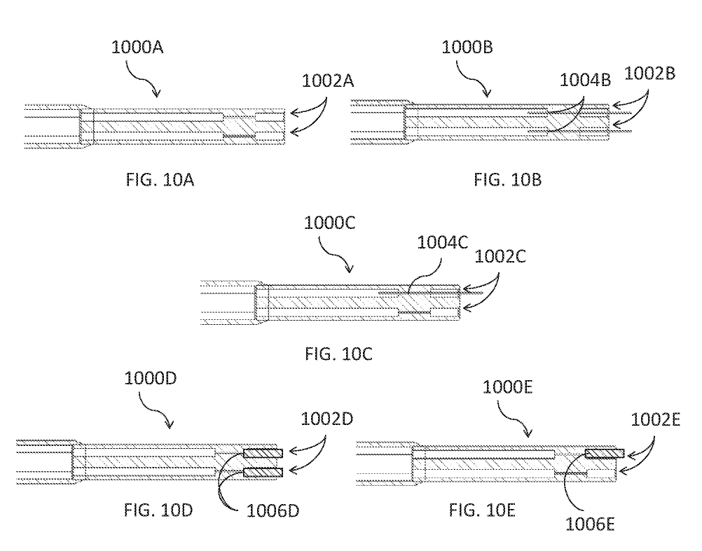

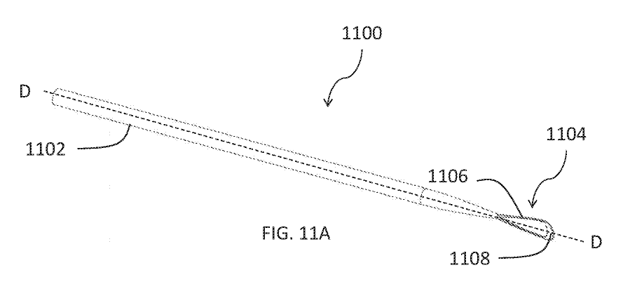

Accordingly, FIGS. 10A-10E illustrate exemplary embodiments of a bifurcated outlet tubular member having channels defined thereby being opened, plugged, occluded/obstructed, etc., in order to adjust the flow resistance imparted to the fluid or the backpressure on the fluid. The bifurcated outlet tubular member in each of FIGS. 10A-10E defines two longitudinally extending channels; similar to the outlet tubular member 132B described herein in reference to FIG. 1C.

In FIG. 10A, a first exemplary embodiment of an outlet tubular member 1000A is provided. As illustrated in FIG. 10A, the outlet tubular member 1000A defines two longitudinally extending channels 1002A. In some aspects, the two longitudinally extending channels 1002A are opened in the outlet tubular member 1000A via an opening apparatus, such as, a laser pulse removing material of the outlet tubular member 1000A, so as to decrease the flow resistance imparted to the fluid or the reduce backpressure on the fluid.

In FIG. 10B, a second exemplary embodiment of an outlet tubular member 1000B is provided. As illustrated in FIG. 10B, the outlet tubular member 1000B defines two longitudinally extending channels 1002B. In some aspects, the two longitudinally extending channels 1002B are partially occluded via an obstructing member in order to increase the flow resistance imparted to the fluid or the increase backpressure on the fluid as compared with FIG. 10A. More particularly, and as illustrated in FIG. 10B, the obstructing member comprises sutures 1004B.

In FIG. 10C, a third exemplary embodiment of an outlet tubular member 1000C is provided. As illustrated in FIG. 10C, the outlet tubular member 1000C defines two longitudinally extending channels 1002C. In some aspects, one of the two longitudinally extending channels 1002C is partially occluded by an obstructing member formed as a suture 1004C, while a second of the two longitudinally extending channels 1002C is opened, having had the relevant suture 1004C pulled out of the second channel, in order to decrease the flow resistance imparted to the fluid or reduce the backpressure on the fluid as compared with FIG. 10B.

FIG. 10D, a fourth exemplary embodiment of an outlet tubular member 1000C is provided. As illustrated in FIG. 10D, the outlet tubular member 1000D defines two longitudinally extending channels 1002D. In some aspects, the two longitudinally extending channels 1002D are totally occluded via obstructing members formed as plugs 1006D. In this manner, the plugs 1006D substantially prevent fluid from flowing out of the outlet tubular member 1000D and thereby significantly increase the flow resistance imparted to the fluid or the backpressure on the fluid as compared with FIGS. 10A, 10B.

In FIG. 10E, a fifth exemplary embodiment of an outlet tubular member 1000E is provided. As illustrated in FIG. 10E, the outlet tubular member 1000E defines two longitudinally extending channels 1002E. In some aspects, one of the two longitudinally extending channels 1002E is fully occluded via an obstructing member formed as a plug 1006E, while a second of the two longitudinally extending channels 1002E is opened, having had the plug 1006E pulled out of the second channel, in order to partially decrease the flow resistance imparted to the fluid or the backpressure on the fluid as compared with FIG. 10D.

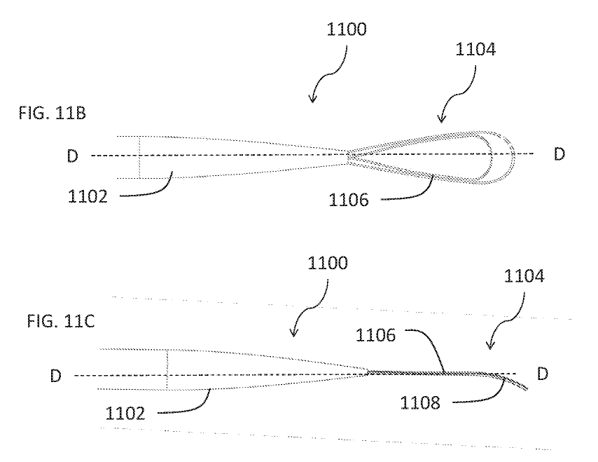

Referring now to FIGS. 11A-15B, exemplary embodiments of manipulating tools for manually positioning the outlet port 134 defined by the distal end of the outlet tubular member 132A, 132B of the pressure-reducing device 100A, 100B. Notably, manipulating tools are desirable where an outlet port 134 is positioned relative to the external surface such that the tubular outlet member 132A, 132B is incapable of having a length, cross-sectional area, etc., adjusted, or is otherwise not conveniently accessible. For example, where the pressure-reducing device 100A, 100B is subconjunctivally implanted in an eye in an anterior chamber, the distal end of the outlet tubular member 132A, 132B lies externally to an ocular surface of the eye, such as the fornix or cul-de-sac region under the eyelid. In order to expose the outlet port 134 from the fornix or cul-de-sac region under the eyelid, in this example, a manipulation tool capable of engaging the outlet tubular member 132A, 132B and selectively positioning the distal end thereof for ready access is utilized.

FIGS. 11A-11C illustrate a first exemplary embodiment of a manipulation tool 1100. The manipulation tool 1100 comprises, in some aspects, a shaft 1102 having a loop arrangement 1104 disposed at a first end of the shaft 1102, the loop arrangement 1104 being configured to be capable of receiving the distal end of the outlet tubular member 132A, 132B therein and to separate the distal end of the outlet tubular member 132A, 132B from the external surface and to move the distal end to an accessible position (e.g., removed from the fornix or cul-de-sac region under the eyelid). In some aspects, the loop arrangement 1104 includes a loop member 1106 having a proximal end extending from the first end of the shaft, the proximal end of the loop member being coplanar with a longitudinal axis D-D of the shaft. In these aspects, the loop member 1106 is configured to receive the outlet tubular member 132A, 132B through a central portion defined by the loop member 1106.

In some aspects, the loop member 1106 extends from the proximal end thereof to a distal end comprising an inclined surface 1108 extending at an obtuse angle away from the proximal end of the loop member 1106. Accordingly, in such instances, the obtuse angle at which the inclined surface 1108 of the loop member 1106 extends from the proximal end thereof corresponds to a curvature of the surface external to the pressurized chamber. For example, the inclined surface 1108 of the loop member 1106 corresponds to the curvature of an eye in which the pressure-reducing device 100A, 100B is implanted.

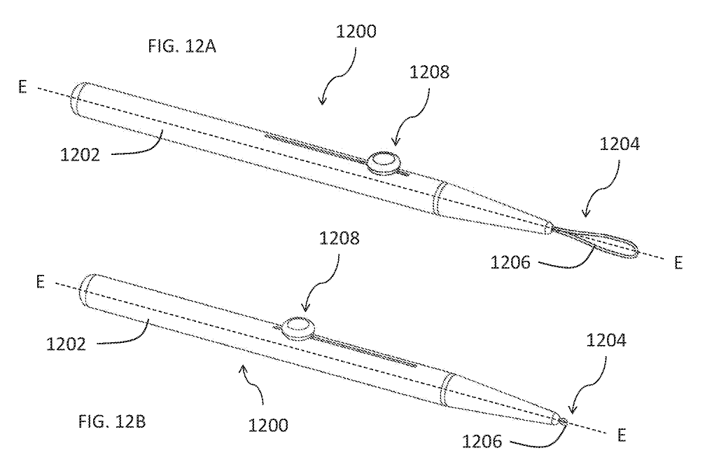

FIGS. 12A-12B illustrate a second exemplary embodiment of a manipulation tool, 1200. The manipulation tool 1200 comprises, in some aspects, a shaft 1202 having a loop arrangement 1204 disposed at a first end of the shaft 1202, the loop arrangement 1204 being configured to be capable of receiving the distal end of the outlet tubular member 132A, 132B therein and to separate the distal end of the outlet tubular member 132A, 132B from the external surface and to move the distal end to an accessible position (e.g., removed from the fornix or cul-de-sac region under the eyelid).

In some aspects, the loop arrangement 1204 includes an adjustable loop member 1206 having a proximal end extending from the first end of the shaft 1202 to an opposed distal end. The adjustable loop member 1206 defines a loop that is configured to receive the outlet tubular member 132A, 132B through a central portion defined by the adjustable loop member 1206. In some aspects, the adjustable loop member 1206 is coupled to an adjustment arrangement 1208 engaged between the shaft 1202 and the proximal end of the adjustable loop member 1206.

More particularly, the adjustment arrangement 1208 is configured to extend or retract the proximal end of the adjustable loop member 1206 with respect to the shaft 1202 so as to alter a size of a loop defined by the adjustable loop member 1206 and to release or secure the distal end of the outlet tubular member 132A, 132B therein. Moving the adjustment arrangement 1208 axially about a longitudinal axis E-E of the shaft 1202 enables retraction and extension of the adjustable loop member 1206.

For example, FIG. 12A illustrates the adjustment arrangement 1208 in a first position in which the proximal end of the adjustable loop member 1206 is in an extended configuration to define a maximum sized loop. In the extended configuration, the maximum sized loop enables easier engagement of the distal end of the outlet tubular member 132A, 132B. Likewise, the maximum sized loop enables release of the distal end of the outlet tubular member 132A, 132B from engagement thereby.

In another example, FIG. 12B illustrates the adjustment arrangement 1208 in a second position in which the proximal end of the adjustable loop member 1206 is in a retracted configuration to define a minimum sized loop. In the retracted configuration, the minimum sized loop is able to retain the distal end of the outlet tubular member 132A, 132B therein to thereby securely manipulate the distal end of the outlet tubular member 132A, 132B.

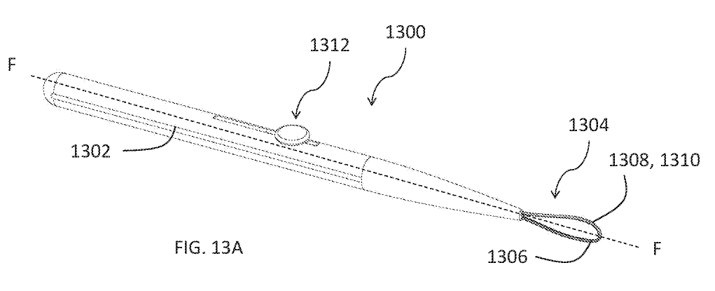

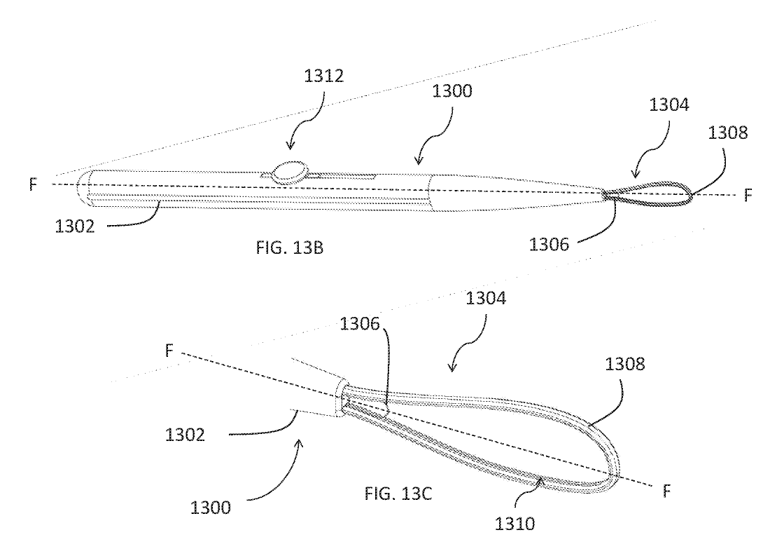

FIGS. 13A-13C illustrate a third exemplary embodiment of a manipulation tool 1300. The manipulation tool 1300 comprises, in some aspects, a shaft 1302 having a loop arrangement 1304 disposed at a first end of the shaft 1302, the loop arrangement 1304 being configured to be capable of receiving the distal end of the outlet tubular member 132A, 132B therein and to separate the distal end of the outlet tubular member 132A, 132B from the external surface and to move the distal end to an accessible position (e.g., removed from the fornix or cul-de-sac region under the eyelid).

In some aspects, the loop arrangement 1304 includes an adjustable loop member 1306 having a proximal end extending from the first end of the shaft 1302 to an opposed distal end. The adjustable loop member 1306 defines a loop that is configured to receive the outlet tubular member 132A, 132B through a central portion defined by the adjustable loop member 1306. As illustrated in FIGS. 13A-13C, the loop arrangement 1304 further comprises a fixed loop member 1308 defining a channel 1310 extending along the fixed loop member 1308 and within a loop defined thereby.

In some aspects, the adjustable loop member 1306 is coupled to an adjustment arrangement 1312 engaged between the shaft 1302 and the proximal end of the adjustable loop member 1306. More particularly, the adjustment arrangement 1312 is configured to extend or retract the proximal end of the adjustable loop member 1306 with respect to the shaft 1302 and the fixed loop member 1308 so as to alter a size of a loop defined by the adjustable loop member 1306 and to release or secure the distal end of the outlet tubular member 132A, 132B therein. Moving the adjustment arrangement 1312 axially along a longitudinal axis F-F of the shaft 1302 enables retraction and extension of the adjustable loop member 1306.

For example, FIG. 13A illustrates the adjustment arrangement 1312 in a first position in which the proximal end of the adjustable loop member 1306 is in an extended configuration to define a maximum sized loop. In these instances, the fixed loop member 1308 is arranged to extend about the maximum sized loop defined by the adjustable loop member 1306 such that the maximum sized loop is received by the channel 1310. In the extended configuration, the maximum sized loop enables easier engagement of the distal end of the outlet tubular member 132A, 132B. Likewise, the maximum sized loop enables release of the distal end of the outlet tubular member 132A, 132B from engagement thereby.

In another example, FIGS. 13B-13C illustrate the adjustment arrangement 1308 in a second position in which the proximal end of the adjustable loop member 1306 is in a retracted configuration to define a minimum sized loop. In the retracted configuration, the minimum sized loop is able to retain the distal end of the outlet tubular member 132A, 132B therein to thereby securely manipulate the distal end of the outlet tubular member 132A, 132B.

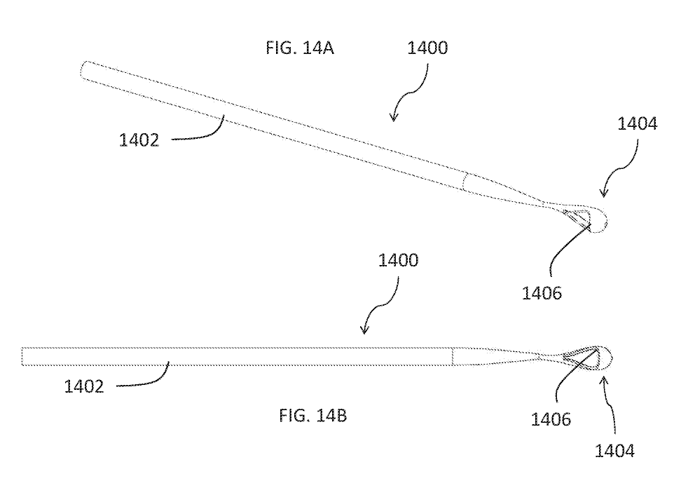

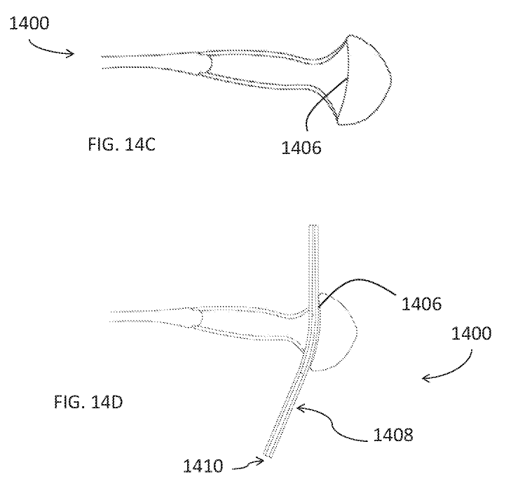

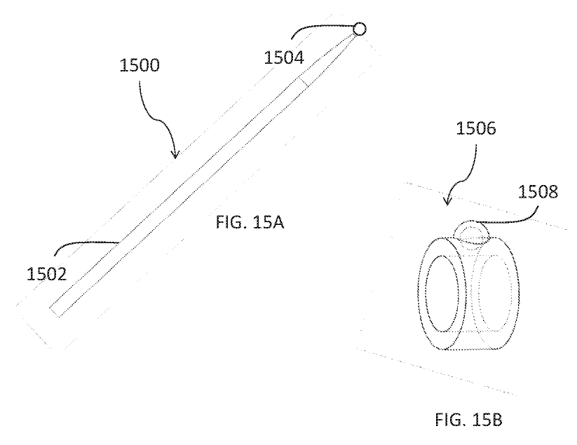

FIGS. 14A-14D illustrate a fourth exemplary embodiment of a manipulation tool 1400. The manipulation tool 1400 comprises, in some aspects, a shaft 1402 having a scoop arrangement 1404 disposed at a first end of the shaft 1402. In such aspects, the scoop arrangement 1404 is configured with a contoured engagement surface 1406 capable of engaging a distal end 1410 of an outlet tubular member 1408 (similar to the outlet tubular member 132A, FIGS. 1A, 1B). The engagement surface 1406 is used to angle the outlet tubular member 1408 downwards for observation. For example, where the external surface is the fornix, the contoured engagement surface 1406 is configured to comfortably travel along the fornix and scoop the distal end 1410 of the outlet tubular member 1408 to thereby separate the distal end 1410 from an external surface and to move the distal end to an accessible position (e.g., removed from the fornix or cul-de-sac region under the eyelid).

FIG. 15A illustrates a fifth exemplary embodiment of a manipulation tool 1500. The manipulation tool 1500 comprises, in some aspects, a shaft 1502 having a magnetized element 1504 engaged with a first end of the shaft 1502. The magnetized element 1504 comprises, in some aspects, iron, nickel, cobalt, lodestone, or any alloy or combination thereof. FIG. 15B illustrates a magnetic sleeve 1506 configured to be engaged with an outlet tubular member (e.g., outlet tubular member 132A, 132B, FIGS. 1A-1C.) More particularly, for example, the magnetic sleeve 1506 is configured to slide over the outlet tubular member either temporarily or otherwise permanently engaged with the outlet tubular member. In some aspects, the magnetic sleeve 1506 is configured to be magnetically attracted to the magnetized element 1504 of the manipulation tool 1500. For example, the magnetic sleeve comprises a metallic element 1508 of iron, nickel, cobalt, lodestone, or any alloy or combination thereof, which is attracted to the magnetized element 1504.

As illustrated in FIG. 15B, the metallic element 1508 is formed as a protrusion extending from a circumferential surface of the magnetized sleeve 1506. Otherwise, the metallic element 1508 is formed as a ring embedded within the magnetic sleeve 1506 or is provided within the outlet tubular member, itself. Accordingly, the magnetized element 1504 of the manipulation tool 1500 is capable of magnetically attracting the metallic element 1508 of the magnetic sleeve 1506, wherein the magnetic sleeve 1506 is engaged with an outlet tubular member, and capable of separating a distal end of the outlet tubular member defining the outlet port from an external surface, such that the distal end is movable to an accessible position (e.g., removed from the fornix or cul-de-sac region under the eyelid).

Embodiments of a manipulation tool other than those described above in reference to FIGS. 11A-15B include a shaft having a porous material at a proximal end thereof, the porous material (e.g., a sponge) being wedge shaped and configured to separate the distal end of the tubular member defining an outlet port from an external surface to move the distal end to an accessible position.

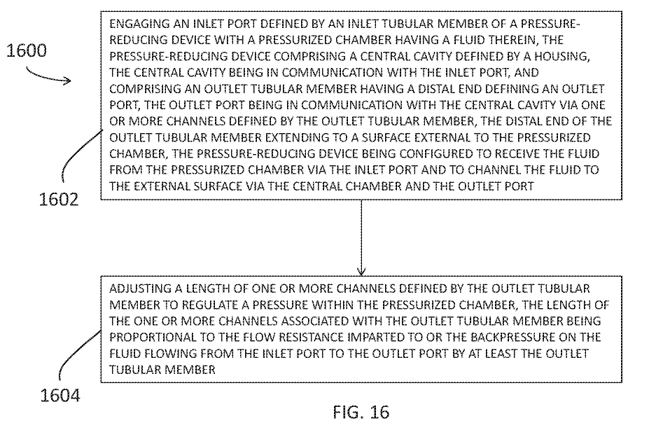

Referring now to FIG. 16, a method for reducing pressure, generally designated 1600, is illustrated. The method 1600 is configured for reducing pressure for many applications, including for example, within an anterior chamber of an eye. In some aspects, a pressure-reducing device (e.g., 100A, 100B, FIGS. 1A-1C) is implanted such that an inlet port is engaged with the pressurized chamber and directs fluid therefrom via an outlet port.

In step 1602, an inlet port defined by an inlet tubular member of a pressure-reducing device is engaged with a pressurized chamber having a fluid therein, the pressure-reducing device comprising a central cavity defined by a housing, the central cavity being in communication with the inlet port, and comprising an outlet tubular member having a distal end defining an outlet port, the outlet port being in communication with the central cavity via one or more channels defined by the outlet tubular member, the distal end of the outlet tubular member extending to a surface external to the pressurized chamber, the pressure-reducing device being configured to receive the fluid from the pressurized chamber via the inlet port and to channel the fluid to the external surface via the central chamber and the outlet port.

In step 1604, the length, constriction, opening, and/or obstruction of the one or more channels defined by the outlet tubular member is adjusted to regulate a pressure within the pressurized chamber, the length of the one or more channels associated with the outlet tubular member being proportional to the flow resistance imparted to or the backpressure on the fluid flowing from the inlet port to the outlet port by at least the outlet tubular member.

Many modifications and other aspects of the disclosure will come to mind to one skilled in the art to which this disclosure pertains having the benefit of the teachings presented in the foregoing descriptions and the associated drawings. Therefore, it is to be understood that the disclosure is not to be limited to the specific aspects disclosed herein and that modifications and other aspects are intended to be included within the scope of the appended claims. Although specific terms are employed herein, they are used in a generic and descriptive sense only and not for purposes of limitation.

What is claimed is:

1. A system for reducing pressure, the system comprising:
a pressure-reducing device comprising an inlet tubular member defining an inlet port, the inlet port being in communication with a central cavity defined by a housing and adapted to engage a pressurized chamber having a fluid therein, and an outlet tubular member having a distal end defining an outlet port, the outlet port being in communication with the central cavity via one or more channels defined by the outlet tubular member, the distal end of the outlet tubular member being adapted to extend to a surface external to the pressurized chamber, the pressure-reducing device receiving the fluid from the pressurized chamber via the inlet port and channeling the fluid to the surface external to the pressurized chamber via the central cavity and the outlet port; and
a cutting tool configured to be capable of engaging the distal end of the outlet tubular member so as to adjust a length of the one or more channels defined by the outlet tubular member to regulate a pressure within the pressurized chamber, the length of the one or more channels being proportional to the flow resistance imparted to or the backpressure on a fluid flowing from the inlet port to the outlet port by at least the outlet tubular member.

2. The system according to claim 1, comprising a manipulation tool configured to be capable of engaging the outlet tubular member so as to selectively position the distal end thereof.

3. The system according to claim 1, comprising a measuring device configured to be capable of engaging the housing or the outlet tubular member for measuring the flow rate of or the backpressure on the fluid flowing from the inlet port to the outlet port.

4. The system according to claim 1, wherein the cutting tool comprises a cut limiter capable of engaging the distal end of the outlet tubular member and a cutting element spaced apart from the cut limiter and capable of engaging the outlet tubular member longitudinally therealong away from the distal end, and for limiting an amount of the length of the one or more channels defined by the outlet tubular member by the cutting tool in one cut.

5. The system according to claim 1, comprising an insertion tool configured to be capable of engaging the distal end of the outlet tubular member so as to introduce an insert into the outlet port, the insert affecting the flow resistance imparted to the fluid or the backpressure on the fluid.

6. The system according to claim 5, wherein the insert is configured to reduce or to increase the flow resistance imparted to the fluid flowing through the outlet port or the backpressure on the fluid within the housing or the outlet tubular member.

7. The system according to claim 1, comprising a compression tool configured to be capable of engaging the outlet tubular member so as to reduce a cross-sectional area of the one or more channels defined thereby, the reduced cross-sectional area increasing the flow resistance imparted to the fluid or increasing the backpressure on the fluid.

8. The system according to claim 1, wherein the length of the one or more channels defined by the outlet tubular member or a cross-sectional area of the one or more channels are configured to be proportional to the flow resistance imparted to the fluid or to the backpressure on the fluid.

9. The system according to claim 2, wherein the manipulation tool comprises a shaft having a loop arrangement disposed at a first end of the shaft, the loop arrangement being configured to be capable of receiving the distal end of the outlet tubular member therein and to separate the distal end of the outlet tubular member from the external surface adjacent thereto.

10. The system according to claim 9, wherein the loop arrangement includes a loop member having a proximal end extending from the first end of the shaft, the proximal end of the loop member being coplanar with a longitudinal axis of the shaft, the loop member extending to a distal end comprising an inclined surface extending at an obtuse angle away from the proximal end of the loop member.

11. The system according to claim 9, wherein the loop arrangement includes an adjustable loop member having a proximal end extending from the first end of the shaft to an opposed distal end, and an adjustment arrangement engaged between the shaft and the proximal end of the adjustable loop member, the adjustment arrangement being configured to extend or retract the proximal end of the adjustable loop member with respect to the shaft so as to alter a size of a loop defined by the adjustable loop member and to release or secure the distal end of the outlet tubular member therein.

12. The system according to claim 11, wherein the loop arrangement comprises a fixed loop member defining a channel extending along the fixed loop member and within a loop defined thereby, the fixed loop member being arranged to extend about the adjustable loop member such that the adjustable loop member in an extended configuration is received by the channel.

13. The system according to claim 2, wherein the manipulation tool comprises a shaft having a magnetized element engaged with a first end of the shaft, the magnetized element being configured to be capable of engaging a magnetic element associated with the distal end of the outlet tubular member defining the outlet port.

14. The system according to claim 4, wherein the cutting tool comprises a guide element aligned with and spaced apart from the cut limiter along an axis, the guide element being configured to receive the distal end of the outlet tubular member therethrough with the distal end extending up to the cut limiter, the cutting element being configured to extend between the guide element and the cut limiter to cut the outlet tubular member along the length thereof.

15. The system according to claim 4, wherein the cut limiter is removable as to remove the limitation on the amount of the length of the outlet tubular member removable by the cutting tool in one cut.

16. The system according to claim 14, wherein the cut limiter and the cutting element are laterally movable relative to the axis in response to actuation by an actuator, the actuator, upon actuation, being configured to laterally move the cut limiter into axial alignment with the guide element prior to laterally moving the cutting element to extend between the guide element and the cut limiter to cut the outlet tubular member along the length thereof.

17. The system according to claim 16, wherein the actuator, upon actuation thereof, is configured to laterally move the cut limiter into axial alignment with the guide element substantially simultaneously with laterally moving the cutting element to extend between the guide element and the cut limiter to cut the outlet tubular member.

18. The system according to claim 5, wherein the insertion tool comprises a cannula having a distal end insertable into the one or more channels defined by the outlet tubular member axially through the outlet port, the cannula being configured to receive the insert therein and being configured to axially move the insert along the cannula and through the distal end thereof to introduce the insert into the one or more channels defined by the outlet tubular member.

19. The system according to claim 18, comprising an adapter mechanism defining a first port configured to be engaged with the distal end of the outlet tubular member and defining an opposing second port configured to receive the cannula, the cannula being configured to introduce the insert received therein through the second port of the adapter mechanism and into the one or more channels defined by the outlet tubular member through the first port of the adapter mechanism.

20. The system according to claim 18, wherein the distal end of the cannula is oblique relative to a longitudinal axis of the cannula.

21. The system according to claim 18, wherein the distal end of the cannula is configured to taper away from a longitudinal axis of the cannula.

22. The system according to claim 18, wherein the cannula defines diametrically opposed longitudinal slots extending from the distal end of the cannula.

23. The system according to claim 1, comprising a tab engaged with the outlet tubular member and spaced apart from the outlet port defined by the outlet tubular member for adjustable positioning of the outlet tubular member.

24. The system according to claim 5, wherein the outlet tubular member defines one or more channels each being configured to receive an insert therein, the insert defining one or more micro-channels extending axially between opposed longitudinal ends of the insert.

25. The system according to claim 24, wherein a radially outermost surface of the insert is longitudinally fluted and defines one or more grooves extending between the opposed longitudinal ends, the one or more grooves cooperating with the one or more channels defined by the outlet tubular member to define flow channels therebetween.

26. The system according to claim 1, comprising bore tool configured to be capable of engaging the outlet tubular member so as to form a hole through a wall of the outlet tubular member along a length thereof, the hole in the wall thereby forming a supplemental outlet port and reducing the length of the one or more channels defined by the outlet tubular member.

27. The system according to claim 1, wherein the outlet tubular member defines two or more channels separated by a bifurcation extending from the distal end of the outlet tubular member, each of the two or more channels being configured to be opened or obstructed.

28. The system according to claim 27, comprising an obstructing member configured to be inserted longitudinally along the outlet tubular member into one or more of the channels defined thereby, the obstructing member laterally extending across the one or more channels so as to increase flow resistance imparted to or backpressure on the fluid flowing through the outlet tubular member.

29. The system according to claim 27, comprising an opening apparatus configured remove material of the outlet tubular member to create the two or more channels and thereby decrease flow resistance imparted to or backpressure on the fluid flowing through the outlet tubular member.

30. The system according to claim 7, wherein the compression tool comprises a compression sheath configured to extend about and compress the outlet tubular member so as to reduce the cross-sectional area of the one or more channels defined thereby.

31. A method for reducing pressure, the method comprising:
engaging an inlet port defined by an inlet tubular member of a pressure-reducing device with a pressurized chamber having a fluid therein, the pressure-reducing device comprising a central cavity defined by a housing, the central cavity being in communication with the inlet port, and comprising an outlet tubular member having a distal end defining an outlet port, the outlet port being in communication with the central cavity via one or more channels defined by the outlet tubular member, the distal end of the outlet tubular member extending to a surface external to the pressurized chamber, the pressure-reducing device being configured to receive the fluid from the pressurized chamber via the inlet port and to channel the fluid to the external surface via the central chamber and the outlet port; and
adjusting a length of the one or more channels defined by the outlet tubular member to regulate a pressure within the pressurized chamber, the length of the one or more channels associated with the outlet tubular member being proportional to the flow resistance imparted to or the backpressure on the fluid flowing from the inlet port to the outlet port by at least the outlet tubular member.

32. The method according to claim 31, wherein adjusting the length of the one or more channels defined by the outlet tubular member comprises actuating a cutting device engaged with the distal end of the outlet tubular member so as to reduce a length of the outlet tubular member, the length of the outlet tubular member being proportional to the flow resistance or the backpressure imparted to a fluid flowing from the inlet port to the outlet port by the housing or by the housing and the outlet tubular member.

33. The method according to claim 31, comprising engaging the outlet tubular member with a manipulation tool so as to selectively position the distal end of the outlet tubular member via manipulation of the manipulation tool.

34. The method according to claim 31, comprising measuring the flow resistance imparted to or the backpressure on the fluid flowing from the inlet port to the outlet port with a measuring device engaged with the housing or the outlet tubular member.

35. The method according to claim 31, comprising introducing an insert into the outlet port, the insert affecting the flow resistance imparted to the fluid or the backpressure on the fluid.

36. The method according to claim 31, comprising adjusting a cross-sectional area of the one or more channels defined by the outlet tubular member, the reduced cross-sectional area increasing the flow resistance imparted to the fluid or increasing the backpressure on the fluid.

37. The method according to claim 31, forming a hole through a wall of the outlet tubular member along a length thereof, the hole in the wall thereby forming a supplemental outlet port and reducing the length of the one or more channels defined by the outlet tubular member.
